(19) United States
(12) Patent Application Publication
(10) Pub. No.: US 2016/0206654 A1
(43) Pub. Date: Jul. 21, 2016

(54) METHODS AND FEED SUPPLEMENTS FOR LIVESTOCK

(71) Applicant: Furst-McNess Company, Freeport, IL (US)

(72) Inventor: Fredrik B. Sandberg, Ankeny, IA (US)

(21) Appl. No.: 15/085,601

(22) Filed: Mar. 30, 2016

Related U.S. Application Data
(63) Continuation-in-part of application No. PCT/US2013/062709, filed on Sep. 30, 2013.
(60) Provisional application No. 62/142,185, filed on Apr. 2, 2015.
(60) Provisional application No. 61/884,695, filed on Sep. 30, 2013.

(51) Int. Cl. A61K 33/34; A61K 31/714; A61K 33/24; A61K 31/07; A61K 31/59; A61K 31/355; A61K 9/16; A61K 36/54; A61K 36/53; A61K 36/81; A61K 36/9066; A61K 36/48; A61K 31/11; A61K 31/192; A61K 31/216; A61K 31/05; A61K 31/165; A61K 31/12; A61K 31/455; A61K 31/51; A61K 31/4188; A61K 31/525; A61K 31/197; A61K 31/4415; A61K 9/00; A23K 50/30; A61K 33/30

(52) U.S. Cl. CPC A61K 33/34; A61K 33/30; A61K 31/714; A61K 33/24; A61K 31/07; A61K 31/59; A61K 31/355; A61K 9/167; A61K 36/54; A61K 36/53; A61K 36/81; A61K 36/9066; A61K 36/48; A61K 31/11; A61K 31/192; A61K 31/216; A61K 31/05; A61K 31/165; A61K 31/12; A61K 31/455; A61K 31/51; A61K 31/4188; A61K 31/525; A61K 31/197; A61K 31/4415; A61K 9/0056; A23K 50/30

(57) ABSTRACT

A feed supplement having a botanical component and a nutritional component. The botanical component comprises cinnamaldehyde, carnosic acid, rosmarinic acid, carvacrol, thymol, capsaicin and curcumin. The nutritional component includes zinc, copper, chromium, and vitamin B. Also provided is a method for improving the nutritional status, such as increasing lean muscle mass, of pigs or other livestock. The method includes feeding to the livestock a composition having a botanical component, including cinnamaldehyde, carnosic acid, rosmarinic acid, carvacrol, thymol, capsaicin and curcumin; and a nutritional component including zinc, copper, chromium, and vitamin B.

### CROSS-REFERENCE TO RELATED APPLICATIONS

This application claims the benefit and priority of U.S. Provisional Patent Application No. 62/142,185, filed Apr. 2, 2015, and is a continuation-in-part of PCT/US2013/062709, filed Sep. 30, 2013, which claims the benefit and priority of U.S. Provisional Patent Application No. 61/884,695, filed Sep. 3, 2013. The entire disclosures of the above applications are incorporated herein by reference.

### BACKGROUND

The development and production of livestock feed is a vital aspect of meeting the nutritional needs of people across the world. Estimates place the revenue for the farm animal food production industry at over $30 billion. Because a major cost of raising livestock is incurred from feeding, supplements are often added to feed in order to provide benefits to animals, such as improved health, growth rates and fat distributions. By improving growth rates and fat distributions, producers of livestock can improve the efficiency of their farms and save costs.

Various supplements can be used depending on the desired results and the age of the animal. For instance, a lactating pig may require a different supplement than a pig that is being prepared for slaughter. Weaning and growing pigs use a majority of the food they intake for growth and maintenance. As pigs mature, a pig's food intake is used primarily for maintenance and reproduction (Sandberg, et al., Br. J. Nutr., 2005, 93:205-212; Sandberg, et al., Br. J. Nutr., 2005, 93:213-224). Digested food that is not used for maintenance or reproduction gets stored in the pig as fat. Consequently, as pigs become physiologically older, at a certain weight, which depends on the pigs' genetics, they start to increase the rate of fat deposition and reduce the rate of protein deposition (Sandberg, et al., Br. J. Nutr., 2005, 93:205-212; Sandberg, et al., Br. J. Nutr., 2005, 93:213-224). The increased rate of fat deposition and reduced rate of protein deposition is apparent even when supplements are added to pig feed. The consequence is that the pigs start to grow more slowly and become less efficient at converting feed into meat. This slower growth and inefficient conversion of feed into meat is most noticeable in pigs with a body weight of about 200 pounds until time for slaughter, which can range from 250 pounds to about 300 pounds.

A variety of additives and nutritional supplements are described in the art for modifying the nutritional and metabolic status of pigs and other livestock. Such additives include nutritional supplements (e.g., vitamins, minerals, and protein supplements), as well as pharmacologic actives (e.g., chemotherapeutics such as beta agonists, and antibiotics). Although pharmacologic actives may be effective to improve the health and metabolism of animals, their use may present regulatory, consumer and welfare concerns. Accordingly, there remains a need to develop feed supplements that increase a pig's feed intake and growth rate, especially during finishing.

### SUMMARY

The present technology provides compositions that are suitable as animal feed or animal feed supplements for consumption by livestock, such as pigs. In various embodiments, the composition is a feed supplement, comprising a botanical component, such as an essential oil from an herb or spice, selected from the group consisting of cinnamon, oregano, chili, turmeric, fenugreek, extracts thereof, and combinations thereof. The compositions also contain one or more of zinc, copper, chromium, and vitamin B. For example, the zinc may be a zinc methionine complex, the copper may be copper chloride, the chromium may be chromium proprionate, and the vitamin B may be vitamin B₁₂.

The present technology further provides methods for improving the nutritional status of a livestock animal, such as increasing the lean muscle mass. Such methods include feeding a livestock animal a food composition comprising a botanical component comprising cinnamaldehyde, carnosic acid, rosmarinic acid, carvacrol, thymol, capsaicin and curcumin; and a nutritional supplement selected from the group consisting of zinc, copper, chromium, vitamin B, and combinations thereof. As described above, the cinnamaldehyde, carnosic acid, rosmarinic acid, carvacrol, thymol, capsaicin and curcumin can be added in purified forms, or as cinnamon, rosemary, oregano, chili peppers, turmeric, or extracts thereof. In various embodiments, the food composition to be fed to the livestock can further comprise vitamin A, vitamin D, and vitamin E. Feeding the food composition can be performed from livestock weaning to finishing, or only during finishing.

### DETAILED DESCRIPTION

The following description of technology is merely exemplary in nature of the subject matter, manufacture and use of one or more inventions, and is not intended to limit the scope, application, or uses of any specific invention claimed in this application or in such other applications as may be filed claiming priority to this application, or patents issuing therefrom. A non-limiting discussion of terms and phrases intended to aid understanding of the present technology is provided at the end of this Detailed Description.

### Compositions

The present technology relates to compositions suitable for use as feed supplements. In various embodiments, the compositions comprise a combination of ingredients that support and promote feed intake in livestock. As used herein, livestock includes any animal kept for commercial useful purposes, such as domesticated animals that are raised for producing commodities such as food (e.g., milk and meat), animal products (e.g., fiber), or working in an agricultural or other commercial activity. Livestock include pigs, cows, horses, sheep, chickens and goats. In a preferred embodiment, the livestock animal is a pig.

In various embodiments, the composition is an animal feed, which is intended to provide essentially all nutritional needs of a livestock animal. Such animal feed compositions comprise a bulk food material, which supplies the animal with energy, protein, amino acids, fat as well as vitamins and minerals, and a feed supplement, as described further herein. The compositions of the present technology provide a diet with ingredients that increase feed intake and promote efficient partitioning of nutrients. For example, the combination of these modes of action can support faster growth rates and greater rates of lean (as well as fat) deposition in late finishing. The compositions support enhanced carcass quality by increasing loin depth and carcass yield, and may reduce variation in weight of carcasses at marketing. Therefore, the compositions are especially useful for increasing the growth rate of pigs with a body weight from about 200 pounds until the pig weighs from about 250 pounds to about 300 pounds, or until the pig is otherwise ready for slaughter. Although many benefits can be obtained by feeding the compositions to livestock for finishing, the compositions can be used at all stages of production from weaning to market.

Further, in various embodiments, the present technology provides compositions that improve the general health and nutritional status of livestock, such as pigs. It has been found that livestock animals that are disease challenged, such as having inflammation, will partition nutrients away from maintenance and growth, towards support of immune functions and repair of damaged tissues (Sandberg, et al., J. Anim. Sci., 2006, 84:1552-1566; Sandberg, et al., Br. J. Nutr., 2005, 93:205-212; Sandberg, et al., Animal, 2007, 1:67-86). Partitioning nutrients away from maintenance and growth causes a reduced efficiency in converting feed into meat (Sandberg, et al., Br. J. Nutr., 2005, 93:205-212; Sandberg, et al., Animal, 2007, 1:67-86). In various embodiments, the compositions of the present technology support the livestock animal's natural desire to grow as efficiently and as quickly as possible to attain its final degree of maturity.

In various embodiments, the compositions of the present invention comprise two or more components (e.g., two or more botanical components, two or more nutritional components, or a combination of botanical components and nutritional components) that afford synergistic benefits in the feeding of animals, present at synergistic levels. As referred to herein, such synergistic combinations comprise components at levels that, when fed to an animal, afford a nutritional benefit greater than the additive effect of feeding each component individually.

### Botanical Component

The compositions of the current technology comprise one or more essential oils, oleoresins or other botanical components. In various embodiments, the compositions comprise botanical components in the form of herbs, spices, essential oils or other extracts thereof, and combinations thereof.

Botanical components among those useful herein are selected from the group consisting of cinnamon, oregano, rosemary, sage, chili, turmeric, fenugreek, extracts thereof, and combinations thereof. These herbs and spices are rich in various components useful in the present compositions. For example, cinnamon is rich in cinnamaldehyde; oregano is rich in carvacrol and thymol; rosemary and sage are rich in carnosic acid and rosmarinic acid; chili is rich in capsaicin; turmeric is rich in curcumin, and fenugreek is rich in diosgenin and trigonelline. Therefore, in addition to the herbs and spices, or in the alternative, the composition can comprise the active ingredients from the herbs and spices, including cinnamaldehyde, carnosic acid, rosmarinic acid, carvacrol, thymol, capsaicin, curcumin, diosgenin, trigonelline and combinations thereof. Such extracts and compounds may be isolated from natural sources using methods among those known in the art (Douglas, et al., Herbs, spices and essential oils. UNIDO and FAO 2005).

For example, the present compositions may contain cinnamon, a cinnamon essential oil, cinnamaldehyde, or combinations thereof. Cinnamon essential oil may be obtained from the bark of cinnamon trees, of the genus Cinnamomum, by methods among those known in the art. Cinnamaldehyde may be made synthetically, or by extraction from cinnamon bark, such as by steam distillation.

Oregano essential oil may be made from the leaves and flowers of the herb, Origanium vulgare. The oregano oil may comprise carvacrol and thymol. Alternatively, carvacrol and thymol can be synthetically produced.

Rosemary oil may be extracted from Rosmarinus officinalis, and may be a source of rosmarinic acid, carnosic acid, or both in the nutritional compositions (Windisch, et al., J. Anion. Sci., 2008, E:140-148). In addition to Origanium vulgare and Rosmarinus officinalis, rosmarinic acid may be extracted from culinary herbs such as Ocimum basilicum (basil), Ocimum tenuiflorum (holy basil), Melissa officinalis (lemon balm), Origanum majorana (marjoram), Salvia officinalis (sage), thyme and peppermint, and Prunella vulgarism, Heliotropium foertherianum, Maranta leuconeura, Maranta depressa, Thalia geniculate, and Anthoceros agrestis. Alternatively, carnosic acid and rosmarinic acid can be individually produced synthetically.

Chili extract containing capsaicin may be obtained from chili peppers of the genus Capsicum. Similarly, turmeric extract, containing curcumin, may be obtained from the leaves of the herb Curcuma longa. Alternatively, the capsaicin and curcumin can be individually synthetically produced.

Without limiting the mechanism, function or utility of the present technology, it is believed that, in various embodiments, the essential oils and oleoresins provide antibacterial, antiviral, anti-inflammatory and other therapeutic properties that are mediated through their natural phyto-nutrient mechanisms. Additionally, compositions of the present technology may stimulate gastric secretions that support increased feed intake, or increase blood flow through vasodilator mechanisms, supporting an increased rate of heat loss from the body.

For example, cinnamaldehyde may have antimicrobial properties (Burt, Int. J. Food Micro., 2004, 94:223-253; Si, et al., J. App. Micro., 2006, 100:296-305), especially with respect to oral bacteria, is a fungicide, is an anti-cancer agent, and stimulates growth (Lee, Essential oils in broiler nutrition. Dissertation, University of Utrecht, The Netherlands. 2002. (ISBN Number: 90-393-3222-3)). Carnosic acid is a potent antioxidant, and may protect skin cells against UV radiation, afford anti-inflammatory properties, and provide protection against carcinogens. Rosmarinic acid is an anxiolytic (inhibits anxiety), antioxidant, and may inhibit Japanese encephalitis (common in pigs), and afford anti-microbial and antioxidant properties (Windisch, et al., J. Anim. Sci., 2008, E:140-148). Rosmarinic acid may also provide anti-cancer properties, slowing or preventing metastasis by inhibiting matrix metallopeptidase 13 (MMP-13) and having an antiproliferative effect on tumor cells by inhibiting Indoleamine-pyrrole 2,3-dioxygenase (IDO), which is an enzyme that prevents the immune system from targeting tumor cells. Inhibiting IDO may also reduce proinflammatory cytokine activity. Carvacrol may inhibit the growth of bacteria (Burt, Int. J. Food Micro., 2004, 94:223-253; Si, et al., J. App. Micro., 2006, 100:296-305), including Escherichia coli, Bacillus cereus, Staphylococcus aureus, Salmonella typhimurium, and Pseudomonas aeruginosa. However, the anti-bacterial properties of carvacrol are minimal in regard to probiotic species found in the gut. By suppressing prostaglandin-endoperoxide synthase 2 (COX-2), carvacrol may also have anti-inflammatory properties. Carvacrol has also been shown to improve feed conversion in animals (Lee, Essential oils in broiler nutrition. (Dissertation, University of Utrecht, The Netherlands. 2002. (ISBN Number: 90-393-3222-3)). Thymol may provide antimicrobial properties, inhibiting bacterial growth, including Aeromoans hydrophila and Staphylococcus aureus, and act as a natural fungicide (Windisch, et al., J. Anim. Sci., 2008, E:140-148). Thymol, may also stimulate growth (Lee, Essential oils in broiler nutrition. Dissertation, University of Utrecht, The Netherlands. 2002. (ISBN Number: 90-393-3222-3)), and protect against ringworm and hookworm infections (Burt, Int. J. Food Micro., 2004, 94:223-253; Si, et al., J. App. Micro., 2006, 100:296-305). Capsaicin may act as an analgesic and a vasodilator, and may have the ability to kill various types of cancer cells by causing apoptosis, and promotes heat loss from the body. Capsaicin also promotes digestion (Lee, Essential oils in broiler nutrition. Dissertation, University of Utrecht, The Netherlands. 2002. (ISBN Number: 90-393-3222-3)). Curcumin may provide antioxidant properties, preventing the growth of various tumors, and boosting the immune system by suppressing the activity of pro-inflammatory mediators. Curcumin may also protect against skin diseases, colitis, viruses, and stomach ulcers, stimulates gastric secretions, and promotes digestion (Lee, Essential oils in broiler nutrition. Dissertation, University of Utrecht, The Netherlands. 2002. (ISBN Number: 90-393-3222-3)). Curcumin may also assist in the maintenance of normal cholesterol levels. Diosgenin is a steroid sapogenin, and may assist in the maintenance of normal cholesterol levels. Trigonelline is an alkaloid which may assist in blood sugar regulation.

### Nutritional Components: Vitamins and Minerals

Compositions of the present technology further comprise one or more vitamins and minerals. In various embodiments, the compositions comprise a mineral selected from the group consisting of zinc, copper, chromium, calcium, magn sodium, selenium, manganese, molybdenum, and mixtures thereof. Preferably, the compositions comprise a mineral selected from the group consisting of zinc, copper, chromium, and combinations thereof.

It is understood that, as formulated, the compositions comprise such minerals in a bioavailable form, such as in a nutritionally-acceptable salt or other compound. Such a “nutritionally-acceptable” compound provides a nutritional benefit, without undue adverse side effects (such as toxicity, irritation, or allergic response), commensurate with a reasonable benefit/risk ratio when used in the manner of this technology. Accordingly, for example, the compositions of the present technology may comprise a zinc methionine complex, copper chloride, and chromium tripicolinate or chromium priopionate.

Without limiting the mechanism, function or utility of the present technology, it is believed that, in various embodiments, a zinc methionine complex, and tri-basic copper chloride may crease growth rates supplementing the livestock with highly digestible forms of the essential nutrients, zinc and copper. Chromium (e.g., chromium proprionate) may help the livestock maintain the natural function of the insulin-IGF-1 growth hormone axis supporting increased deposition of nutrients s body lean and fat.

The compositions of the present technology also comprise a vitamin, selected from the group consisting of vitamin A, vitamin B, vitamin D, vitamin E, vitamin K and combinations thereof. The vitamin A can be in the form of retinol, retinyl acetate, palmitate, alpha-carotene, beta-carotene, gamma-carotene, beta-cryptoxanthin, or combinations thereof. The vitamin B can be vitamin B₁ (thiamine, or thiamine mononitrate), vitamin B₂ (riboflavin), vitamin B₃ (niacin, or niacinamide), vitamin B₅ (pantothenic acid, or calcium pantothenate), vitamin B₆ (pyridoxine, pyridoxine hydrochloride, pyridoxal, pyridoxamine, or pyridoxine hydrochloride), vitamin B₇ (biotin or d-biotin), vitamin B₉ (folic acid), vitamin B₁₂ (cobalamins or cyanocobalamin), or combinations thereof. The vitamin D can be in the form of vitamin D₁ (molecular compound of ergocalciferol with lumisterol, 1:1), vitamin D₂ (ergocalciferol), vitamin D₃ (cholecalciferol), vitamin D₄ (22-dihdroergocalciferol), vitamin D₅ (sitocalciferol), or combinations thereof. In various embodiments, the composition includes tocopherols, such as alpha-tocopherol (vitamin E), beta-tocopherol, gamma-tocopherol, delta-tocopherol; tocotrienols (also referred to as “tocols”), such as alpha-tocotrienol, beta-tocotrienol, gamma-tocotrienol, delta-tocotrienol; or combinations thereof. The vitamin K can be vitamin K₃ (menadione dimethylpyrimidinol bisulfite).

Preferably the compositions comprise vitamin B. For example, compositions may comprise vitamin A, vitamin D, vitamin E, vitamin B₁₂, riboflavin, niacin, calcium pantothenate, menadione dimethylpyrimidinol bisulfite, folic acid, pyridoxine hydrochloride, thiamine mononitrate, and d-biotin. Without limiting the mechanism, function or utility of the present technology, it is believed that, in various embodiments, compositions provide high levels of vitamin A and D to support optimum growth, and a combination of B-vitamins to support metabolism and muscle deposition.

### Optional Components

The compositions of the present technology may comprise optional components, including additional nutritional components (such as amino acids), absorption enhancing components, digestive aids (such as enzymes and probiotics), flavorants (artificial and natural flavor components), preservatives, and formulation aids.

For example, the compositions of the present technology may optionally comprise probiotic bacteria. Non-limiting examples of probiotic bacteria include Bacillus Subtilis and Bacillus Lichenformis. Both of these bacteria secrete enzymes that breakdown fibrous materials in the gastrointestinal tract of livestock. The activity of the bacteria continues after excretion in the feces, breaking down materials on slats, in pits or in lagoons. The probiotic bacteria also reduce gaseous ammonia excretions by at least about 30% and hydrogen sulfide excretions by at least about 15% by reducing fermentation activity in the hindgut of livestock.

Plant substrates are composed of multiple fractions of starch, fibers, and non-starch polysaccharides, as well as proteins. By providing multiple enzyme activities in a purified form, together with providing bacteria that will continue to produce enzyme activities in the stomach and intestines of livestock, feed ingredients are digested more efficiently. By digesting feed ingredients more efficiently (for example, in pigs of all degrees of maturity, including weaned pigs to finishing pigs to sows), the energy value of the feed consumed is increased. Animals that can digest their feed more effectively may also have higher rate of passage of feed through the gastrointestinal tract, leading to a higher rate of feed intake, even whilst consuming diets relatively high in fibrous materials.

Further components can be added to the composition to provide supplemented feed suitable for lactating livestock to support the essential amino acid and energy requirements of, for example, high producing sows, specifically for first and second parity sows, to maximize life time performance, by preventing excessive weight loss during lactations. Therefore, in various embodiments, the present technology provides for the addition of extruded soybean meal, which is a source of highly digestible protein and fat, and soybean meal, which is a source of highly digestible protein. The compositions may also comprise coconut oil, dried whey solids, dried whey protein concentrate, soy lecithin, and cereal food fines. Coconut oil is a source of digestible protein, dried whey protein concentrate is a source of digestible protein, soy lecithin aids in the digestion of fats by breaking down larger fat globules into smaller fat globules, and cereal food fines are a source of multiple sugars. Further, the amino acids L-valine, D1-methionine supplement, L-threonine, L-lysine monohydrochloride, and tryptophan, can be added to complete a highly specific amino acid balance of the composition. A correct balance of amino acids is particularly important for livestock during lactation, especially for sows.

### Formulation

The compositions of the present technology comprise one or more botanical components (e.g., herbs, spices, essential oils, and oleoresins), at nutritionally-acceptable levels. Exemplary levels of components are set forth in the following table, in reference to a feed supplement composition of the present technology.

| Component | Level |
|---|---|
| Cinnamon | from about 0.01% (wt) to about 50% (wt) |
| | from about 1% (wt) to about 25% (wt) |
| | about 3% (wt) |
| Oregano | from about 0.01% (wt) to about 50% (wt) |
| | from about 1% (wt) to about 25% (wt) |
| | about 5% (wt) |
| Chili | from about 0.01% (wt) to about 15% (wt) |
| | from about 1% (wt) to about 10% (wt) |
| | about 2% (wt) |
| Turmeric | from about 0.01% (wt) to about 90% (wt) |
| | from about 1% (wt) to about 50% (wt) |
| | about 4% (wt) |
| Fenugreek | from about 1% (wt) to about 95% (wt) |
| | from about 10% (wt) to about 80% (wt) |
| | about 70% (wt) |
| Zinc | from about 5,000 ppm to about 20,000 ppm |
| | from about 10,000 ppm to about 15,000 ppm |
| | about 12,000 ppm |
| Chromium | from about 30 ppm to about 120 ppm |
| | from about 50 ppm to about 100 ppm |
| | about 80 ppm |
| Copper | from about 10,000 ppm to about 50,000 ppm |
| | from about 15,000 ppm to about 40,000 ppm |
| | about 30,000 ppm |
| Vitamin B₁₂ | from about 200 mcg/pound to about 800 mcg/pound |
| | from about 300 mcg/pound to about 600 mcg/pound |
| | about 500 mcg/pound |
| Riboflavin | from about 30 ppm to about 500 ppm |
| | from about 50 ppm to about 150 ppm |
| | about 120 ppm |
| Niacin | from about 200 ppm to about 5000 ppm |
| | from about 600 ppm to about 1,000 ppm |
| | about 800 ppm |
| d-Panothenic Acid | from about 100 ppm to about 2000 ppm |
| | from about 300 ppm to about 500 ppm |
| | about 480 ppm |
| Folic Acid | from about 30 ppm to about 500 ppm |
| | from about 50 ppm to about 150 ppm |
| | about 80 ppm |
| Pyridoxine | from about 30 ppm to about 500 ppm |
| | from about 50 ppm to about 150 ppm |
| | about 72 ppm |
| Thiamine | from about 30 ppm to about 500 ppm |
| | from about 50 ppm to about 150 ppm |
| | about 72 ppm |
| Vitamin A | from about 50,000 IU/lb to about 500,000 IU/lb |
| | from about 100,000 IU/lb to about 300,000 IU/lb |
| | about 200,000 IU/lb |
| Vitamin D | from about 1,000 IU/lb to about 100,000 IU/lb |
| | from about 5,000 IU/lb to about 30,000 IU/lb |
| | about 18,000 IU/lb |
| Vitamin E | from about 100 IU/lb to about 10,000 IU/lb |
| | from about 500 IU/lb to about 3,000 IU/lb |
| | about 1,200 IU/lb |

In various embodiments, the feed composition comprises a botanical component:nutritional component ratio of from about 1:50 to about 90:1. The botanical component:nutritional component ratio can be about 1:50, about 2:50 (about 1:25), about 5:50 (about 1:10), about 10:50 (about 1:5), about 15:50 (about 3:10), about 20:50 (about 2:5), about 25:50 (about 1:2), about 30:50 (about 3:5), about 35:50 (about 7:10), about 40:50 (about 4:5), about 45:50 (about 9:10), about 50:50 (about 1:1), about 2:1, about 4:1, about 6:1, about 8:1, about 10:1, about 12:1, about 14:1, about 16:1, about 18:1, about 20:1, about 30:1, about 40:1, about 50:1, about 60:1, about 70:1, about 80:1, or about 90:1.

In various embodiments, the feed supplement of the present technology comprises a carrier, to facilitate mixing with a base feed composition and effective delivery to the livestock animal. Such carriers include those known in the art. For example, in some embodiments a carrier comprises finely ground distillers dried grains with solubles (DDGS), rice hulls, calcium carbonate, and combinations thereof. DDGS is commercially available, such as SOLULAC®, sold by Grain Processing Corp., Muscatine, Iowa. The feed supplements may optionally comprise mineral oil and/or vegetable or animal derived oils for fat soluble ingredients, as a feed additive and to reduce dustiness.

In various embodiments, the compositions consist essentially of one or more botanical components (or herbs or spices containing such essential oils or oleoresins), vitamins, minerals, and one or more optional ingredients as described above. In general, compositions of the present technology may optionally comprise antibiotics or other chemotherapeutic actives. However, in some embodiments, preferably the compositions do not comprise antibiotics or other chemotherapeutic agents, such as ractopamine. In some embodiments, the compositions are essentially free, or do not contain, carnitine. Thus, for example, the level of carnitine in some compositions is less than about 5 ppm, preferably less than 1 ppm.

The present technology also provides animal food compositions comprising a bulk feed material and a feed supplement of the present technology. Bulk food material useful in this technology includes materials known in the art for feeding to livestock. For example, the bulk food material may contain grains, such as corn, sorghum, wheat, rye, triticale, oats, barley and combinations thereof. Feed compositions may comprise a bulk feed material and a feed supplement of the present technology, at levels of from about 0.5 to about 15, from about 1 to about 10, or from about 2 to about 7 pounds of the feed supplement per ton of feed composition.

### Methods

The present technology further relates to methods for improving the nutritional status of a livestock animal, such as a pig, by feeding a composition of the present technology to the livestock animal. Methods provided by the present technology include methods for increasing the lean muscle mass of livestock, methods for improving livestock yield, methods of improving parity, methods for improving heat tolerance, methods for reducing still-born livestock, methods for improving food intake, methods for increasing lactation, methods for increasing livestock longevity. Such methods comprise feeding to the livestock animal a nutritionally effective amount of a composition of the present technology.

The present technology also provides methods for managing the nutritional status of a group of livestock animals, such as pigs, by feeding the animals a composition of the present technology. Such methods include methods for improving livestock yield, methods for improving food intake, methods for improving feeding efficiency, and methods for reducing livestock death. Methods also include methods for increasing total yield of full value pigs. Value may be determined by achieving high premiums according to packer matrices.

Thus, for example, the present technology provides methods for improving the nutritional status of a livestock animal comprising feeding the animal a feed supplement, or an animal feed comprising a feed supplement, comprising cinnamaldehyde, carnosic acid, rosmarinic acid, carvacrol, thymol, capsaicin, curcumin, diosgenin, trigonelline, and combinations thereof; and a nutritional component selected from the group consisting of zinc, copper, chromium, vitamin B, and combinations thereof. In various embodiments, the botanical component comprises a mixture of cinnamaldehyde, carnosic acid, rosmarinic acid, carvacrol, thymol, capsaicin, curcumin. As described above, the cinnamaldehyde, carnosic acid, rosmarinic acid, carvacrol, thymol, capsaicin and curcumin can be added in purified forms, or as cinnamon, rosemary, oregano, chili peppers, turmeric, extracts thereof, or combinations thereof. In various embodiments, the food composition to be fed to the livestock can further comprise vitamin A, vitamin D, and vitamin E. Additionally, the food composition can include the various optional components described above.

Feeding the feed supplement can be performed from livestock weaning to finishing, or only during finishing. For examples, the food composition can be fed to pigs from weaning, until time for slaughter. In some embodiments, the feed supplement is fed to pigs from when the pig has a body weight of about 200 pounds until time for slaughter. The feed supplement may also be fed to lactating sows.

Without limiting the mechanism, function or utility of the present technology, it is believed that, in various embodiments, feeding a feed supplement of the current technology, in an animal feed also comprising a bulk food material, increases the feed intake of the livestock by from about 2% to about 20%, relative to the same livestock eating a bulk food material feed without the feed supplement. In some embodiments, feeding the feed supplement increases the feed intake of livestock by from about 8% to about 12%. In some embodiments, the feed intake is increased by from about 5% to about 15%, or from about 6% to about 10%, or from about 8% to about 12%. Additionally, feeding the feed supplement to livestock may increase the growth rate of the livestock by from about 2% to about 20%, relative to the same livestock eating a bulk food material without the feed supplement. In various embodiments, feeding the feed supplement increases the growth rate of livestock by from about 8% to about 12%.

In various embodiments, feeding the feed supplement of the present technology to livestock provides further benefits including improving feed efficiency by from about 0.5% to about 5%, by from about 1% to about 3%. Feeding the feed supplement to livestock may also increase loin depth by from about 0.1 to about 0.3 inches, or by from about 3% to about 10%. Feeding the feed supplement to livestock may increase fat depth by from about 0.05 inches to about 0.1 inches, or by from about 7% to about 14%. Additionally, feeding the feed supplement to livestock may increase the percent lean by from about 0.5% to about 0.75%, and the yield by from about 0.5% to about 1.0%. Feeding the feed supplement may increases total percentile of full value pigs that achieve the highest premiums according to packer matrixes by from about 3% to about 4%. Feeding the feed supplement to livestock may also reduce the number of dead, diseased or disabled animals by from about 0.05% to about 1.0%.

Embodiments of the present technology are further illustrated through the following non-limiting example.

### Example

An exemplary feed supplement of the present technology is made by weighing an amount of the following components: cinnamon, oregano, chili, turmeric, fenugreek, zinc methionine complex, copper chloride, chromium proprionate, vitamin B₁₂ supplement, vitamin A supplement, vitamin D supplement, vitamin E supplement, riboflavin supplement, niacin supplement, calcium pantothenate, menadione dimethylpyrmidinol bisulfite, folic acid, pyridoxine hydrochloride, thiamine mononitrate, d-biotin, mineral oil, and natural and artificial flavors. The components are placed in a mixer along with a carrier comprising DDGS, rice hulls and calcium carbonate. The components and carrier are mixed for about 1 minute to provide a mixture. Mineral oil is added to the mixture and it is mixed for a further 6 minutes to generate the feed supplement. The feed supplement is then packaged in bags.

The composition has the following nutritional profile.

| Component | Level | |
|---|---|---|
| Dry Matter | 92% | |
| Crude Protein | 14% | |
| Total Fat | 5% | |
| Crude Fiber | 5% | |
| Calcium | 0.08% | |
| Phosphorus | 0.36% | |
| Phosphorus (available) | 0.28% | |
| Calcium:Phosphorus ratio | 0.22 | |
| Sodium | 0.29% | |
| Chlorine | 0.99% | |
| Magnesium | 0.09% | |
| Potassium | 0.22% | |
| Sulfur | 0.17% | |
| Zinc | 12,000 | ppm |
| Manganese | 13 | ppm |
| Iron | 131 | ppm |
| Copper | 29,000 | ppm |
| Cobalt | 0.1 | ppm |
| Chromium | 80 | ppm |
| Zinc, organic | 12,000 | ppm |
| Vitamin A | 200,000 | IU/lb |
| Vitamin D | 18,000 | IU/lb |
| Vitamin E | 1,200 | IU/lb |
| Vitamin K-Menadione | 55 | Mg/lb |
| Vitamin B₁₂ | 480 | mcg/lb |
| Riboflavin | 120 | Mg/lb |
| Niacin | 800 | Mg/lb |
| d-Pantothenic Acid | 480 | Mg/lb |
| Folic Acid | 80 | Mg/lb |
| Pyridoxine | 72 | Mg/lb |
| Thiamine | 72 | Mg/lb |
| Biotin | 11.2 | Mg/lb |

| | |
|---|---|
| Methionine + Cystine | .77% |
| Tryptophan | .09% |
| Cystine | .55% |
| Lysine | .39% |
| Methionine | .22% |
| Threonine | .51% |

The feed supplement is removed from a bag and admixed with a bulk feed material so that 5 pounds of the composition are mixed with one ton of the bulk feed material. The resulting animal feed composition is fed to pigs weighing approximately 200 pounds, daily, until the animals reach a weight of from about 260 to about 290 pounds. Relative to equivalent animals of equivalent starting weight fed animal feed not comprising the feed supplement, the animals are found to have increased feed intake of from 8% to 12%, increased growth rate of from 8% to 12%, slightly increased feed efficiency (up to 3%), increased loin depth of from 0.1 to 0.3 inches (3% to 10%), increased fat depth of from 0.05 to 0.1 inches (7% to 14%), increased percent lean of 0.5% to 0.75% points, increased yield of from 0.5% to 1% points, and increased total percentage of full value pigs achieving the highest premiums according to packer matrices by 3 to 4% points. In addition the number of sick or dead pigs is reduced by 0.5 to 1%.

### Non-Limiting Discussion of Terminology

The headings (such as “Introduction” and “Summary”) and sub-headings used herein are intended only for general organization of topics within the present disclosure, and are not intended to limit the disclosure of the technology or any aspect thereof. In particular, subject matter disclosed in the “Introduction” may include novel technology and may not constitute a recitation of prior art. Subject matter disclosed in the “Summary” is not an exhaustive or complete disclosure of the entire scope of the technology or any embodiments thereof. Classification or discussion of a material within a section of this specification as having a particular utility is made for convenience, and no inference should be drawn that the material must necessarily or solely function in accordance with its classification herein when it is used in any given composition.

The description and specific examples, while indicating embodiments of the technology, are intended for purposes of illustration only and are not intended to limit the scope of the technology. Equivalent changes, modifications and variations of specific embodiments, materials, compositions and methods may be made within the scope of the present technology, with substantially similar results. Moreover, recitation of multiple embodiments having stated features is not intended to exclude other embodiments having additional features, or other embodiments incorporating different combinations of the stated features. For example, a component which may be A, B, C, D or E, or combinations thereof, may also be defined, in some embodiments, to be A, B, C, or combinations thereof. Specific examples are provided for illustrative purposes of how to make and use the compositions and methods of this technology and, unless explicitly stated otherwise, are not intended to be a representation that given embodiments of this technology have, or have not, been made or tested.

As used herein, the words “prefer” or “preferable” refer to embodiments of the technology that afford certain benefits, under certain circumstances. However, other embodiments may also be preferred, under the same or other circumstances. Furthermore, the recitation of one or more preferred embodiments does not imply that other embodiments are not useful, and is not intended to exclude other embodiments from the scope of the technology.

As used herein, the word “include,” and its variants, is intended to be non-limiting, such that recitation of items in a list is not to the exclusion of other like items that may also be useful in the materials, compositions, devices, and methods of this technology. Similarly, the terms “can” and “may” and their variants are intended to be non-limiting, such that recitation that an embodiment can or may comprise certain elements or features does not exclude other embodiments of the present technology that do not contain those elements or features.

Although the open-ended term “comprising,” as a synonym of non-restrictive terms such as including, containing, or having, is used herein to describe and claim embodiments of the present technology, embodiments may alternatively be described using more limiting terms such as “consisting of” or “consisting essentially of.” Thus, for any given embodiment reciting materials, components or process steps, the present technology also specifically includes embodiments consisting of, or consisting essentially of, such materials, components or processes excluding additional materials, components or processes (for consisting of) and excluding additional materials, components or processes affecting the significant properties of the embodiment (for consisting essentially of), even though such additional materials, components or processes are not explicitly recited in this application. For example, recitation of a composition or process reciting elements A, B and C specifically envisions embodiments consisting of, and consisting essentially of, A, B and C, excluding an element D that may be recited in the art, even though element D is not explicitly described as being excluded herein. Further, as used herein the term “consisting essentially of” recited materials or components envisions embodiments “consisting of” the recited materials or components.

A” and “an” as used herein indicate “at least one” of the item is present; a plurality of such items may be present, when possible. “About” when applied to values indicates that the calculation or the measurement allows some slight imprecision in the value (with some approach to exactness in the value; approximately or reasonably close to the value; nearly). If, for some reason, the imprecision provided by “about” is not otherwise understood in the art with this ordinary meaning, then “about” as used herein indicates at least variations that may arise from ordinary methods of measuring or using such parameters.

As referred to herein, ranges are, unless specified otherwise, inclusive of endpoints and include disclosure of all distinct values and further divided ranges within the entire range. Thus, for example, a range of “from A to B” or “from about A to about B” is inclusive of A and of B. Further, the phrase “from about A to about B” includes variations in the values of A and B, which may be slightly less than A and slightly greater than B; the phrase may be read be “about A, from A to B, and about B.” Disclosure of values and ranges of values for specific parameters (such as temperatures, molecular weights, weight percentages, etc.) are not exclusive of other values and ranges of values useful herein.

It is also envisioned that two or more specific exemplified values for a given parameter may define endpoints for a range of values that may be claimed for the parameter. For example, if Parameter X is exemplified herein to have value A and also exemplified to have value Z, it is envisioned that Parameter X may have a range of values from about A to about Z. Similarly, it is envisioned that disclosure of two or more ranges of values for a parameter (whether such ranges are nested, overlapping or distinct) subsume all possible combination of ranges for the value that might be claimed using endpoints of the disclosed ranges. For example, if Parameter X is exemplified herein to have values in the range of 1-10, or 2-9, or 3-8, it is also envisioned that Parameter X may have other ranges of values including 1-9, 1-8, 1-3, 1-2, 2-10, 2-8, 2-3, 3-10, and 3-9.

What is claimed is:

1. A feed supplement, comprising:
(a) a botanical component comprising cinnamaldehyde, carnosic acid, rosmarinic acid, carvacrol, thymol, capsaicin and curcumin;
(b) zinc;
(c) copper;
(d) chromium; and
(e) vitamin B.

2. The supplement of claim 1, wherein the botanical component is selected from the group consisting of cinnamon, oregano, chili, turmeric, fenugreek, extracts thereof, and combinations thereof.

3. The supplement of claim 1, wherein the zinc is zinc methionine, the copper is copper chloride, and the chromium is chromium proprionate.

4. The supplement of claim 1, wherein the vitamin B is vitamin B₁ (thiamine, or thiamine mononitrate), vitamin B₂ (riboflavin), vitamin B₃ (niacin or niacinamide), vitamin B₅ (pantothenic acid, or calcium pantothenate), vitamin B₆ (pyridoxine, pyridoxine hydrochloride, pyridoxal, or pyridoxamine, or pyridoxine hydrochloride), vitamin B₇ (biotin), vitamin B₉ (folic acid), vitamin B₁₂ (cobalamins or cyanocobalamin), or combinations thereof.

5. The supplement of claim 1, further comprising vitamin A, vitamin D, vitamin E, or combinations thereof.

6. The supplement of claim 1, wherein the essential oil, zinc, copper, chromium, and vitamin B are blended on a carrier, wherein the carrier comprises dried distillers grains with solubles (DDGS), rice hulls, calcium carbonate, and mixtures thereof.

7. The supplement of claim 1, wherein the supplement further comprises a probiotic.

8. An animal feed comprising a bulk food material and a feed supplement according to claim 1, wherein the bulk food material comprises a grain selected from the group consisting of grains, such as corn, sorghum, wheat, rye, triticale, oats, barley and combinations thereof.

9. A method for increasing the lean muscle mass of a pig, comprising feeding a feed supplement of claim 1.

10. A method for improving the nutritional status of a livestock animal, comprising feeding to the animal a feed composition comprising:
(a) a botanical component comprising cinnamaldehyde, carnosic acid, rosmarinic acid, carvacrol, thymol, capsaicin, and curcumin; and
(b) a nutritional component selected from the group consisting of zinc, copper, chromium, vitamin B, and combinations thereof.

11. The method of claim 10, wherein the botanical component is selected from the group consisting of cinnamon, oregano, chili, turmeric, fenugreek, extracts thereof, and combinations thereof.

12. The method according to claim 10, wherein the composition further comprises vitamin A, vitamin D, and vitamin E.

13. A method for increasing the lean muscle mass of a livestock animal, by feeding the animal according to a method of claim 10.

14. A method for improving the food intake of a livestock animal, by feeding the animal according to a method of claim 10.

15. A method of improving the nutritional status of a pig, according to the method of claim 10.

16. The method of claim 15, wherein the pig has a body weight of at least about 200 pounds.

17. The method of claim 15, wherein the feed supplement increases the feed intake of pigs by from about 5% to about 20% when consumed regularly by the pigs.

18. A feed supplement, comprising:
(a) a botanical component comprising from about 0.01% (wt) to about 50% (wt) cinnamon, from about 0.01% (wt) to about 50% (wt) oregano, from about 0.01% (wt) to about 15% (wt) chili, from about 0.01% (wt) to about 90% (wt) turmeric, and from about 1% (wt) to about 80% (wt) fenugreek;
(b) from about 5000 ppm to about 20,000 ppm zinc;
(c) from about 10,000 ppm, to about 50,000 ppm copper;
(d) from about 30 ppm to about 120 ppm chromium;
(e) from about 50,000 to about 500,000 IU/lb vitamin A;
(f) from about 1000 to about 100,000 IU/lb vitamin D;
(g) from about 100 to about 10,000 IU/lb alpha tocopherol (vitamin E); and
(h) vitamin B.

19. The feed supplement according to claim 18, wherein the vitamin B comprises from about 200 mcg/lb to about 800 mcg/lb vitamin B₁₂, from about 30 ppm to about 500 ppm riboflavin, from about 200 ppm to about 5000 ppm niacin, from about 100 ppm to about 2000 ppm pantothenic acid, from about 30 ppm to about 500 ppm folic acid, from about 30 ppm to about 500 ppm pyridoxine, from about 30 ppm to about 500 ppm thiamine, or combinations thereof.

20. The feed supplement according to claim 18, further comprising from about 5,000 to about 20,000 ppm zinc, from about 30 to about 120 ppm chromium, and from about 10,000 ppm to about 50,000 ppm copper.
